(19) United States
(12) Patent Application Publication
(10) Pub. No.: US 2024/0013395 A1
(43) Pub. Date: Jan. 11, 2024

(54) JOINT SPACE QUANTIFICATION USING 3D IMAGING

(71) Applicant: Arizona Board of Regents on Behalf of the University of Arizona, Tucson, AZ (US)

(72) Inventor: Zong-Ming LI, Tucson, AZ (US)

(21) Appl. No.: 18/218,968

(22) Filed: Jul. 6, 2023

Related U.S. Application Data
(60) Provisional application No. 63/358,548, filed on Jul. 6, 2022.

(51) Int. Cl. G06T 7/00; G16H 50/20

(52) U.S. Cl. CPC G06T 7/0014; G16H 50/20; G06T 2207/20081; G06T 2207/20084; G06T 2207/30008

(57) ABSTRACT

In order to more accurately and precisely diagnose conditions affecting joint spacing, a joint space quantification system is disclosed that identifies each bone in a three-dimensional medical image, generates a three-dimensional computer model that includes a three-dimensional representation of each bone, and identifies bone distances (e.g., shortest distances, centroid distances, etc.) between each three-dimensional representation. The joint space quantification system may then identify conditions affecting joint spacing (and quantify the severity of those conditions), for example by comparing the identified bone distances to previous bone distances of the patient and/or the bone distances of patients diagnosed with conditions affecting joint spacing. In some embodiments, the joint space quantification system also includes a neural network that combines those bone distances with biological, biomechanical, and/or performance data to generate a multivariate model for identifying, predicting, and/or avoiding those conditions affecting joint spacing.

### CROSS-REFERENCE TO RELATED APPLICATIONS

This application claims priority to U.S. Prov. Pat. Appl. No. 63/358,548, filed Jul. 6, 2022, which is hereby incorporated by reference.

### FEDERAL FUNDING

None

### BACKGROUND

The bones of healthy individuals are constrained to certain positions. However, the joint spacing between bones may change if an individual is suffering from certain conditions. Arthritis causes joint spacing to decrease as the amount of cartilage separating the person's bones decreases. Carpal tunnel syndrome describes a narrowing of the carpal tunnel between the carpal bones and the ligament at the top of the tunnel. Ligament injuries cause the spacing between some bones to increase (as those injured ligaments no longer constrain those bones) while, in some instances, compressing the spacing between other bones.

While existing medical imaging technology enables practitioners to view those joint spaces and subjectively evaluate them, existing medical imaging technology does not quantify those bone distances. X-rays, for instance, provide only a two-dimensional image along a single axis. While computed tomography (CT) scans or magnetic resonance images (Mills) are three-dimensional, practitioners use those images to diagnose conditions by looking at the images and subjectively assessing the joint spacing of the patient.

As a result, conditions affecting joint spacing are diagnosed subjectively and the severity of those conditions are diagnosed qualitatively. For instance, ligament injuries are subjectively diagnosed as a mild ligament tear (grade 1), a moderate ligament tear (grade 2), or a complete ligament tear (grade 3). Similarly, using the Kellgren and Lawrence system for classification of osteoarthritis, a practitioner may characterize arthritis as grade 1 (doubtful) if the practitioner subjectively believes that osteophytic lipping is possible but joint space narrowing is doubtful, as grade 2 (minimal) if the practitioner subjectively believes that osteophytes are definite and joint space narrowing is possible, or grade 3 (moderate) if the practitioner subjectively believes that multiple osteophytes are moderate, narrowing of joint space and some sclerosis is definite, and deformity of the bone ends is possible.

Quantifying the joint spacing between bones would enable practitioners to diagnose conditions affecting joint spacing earlier and more accurately and enable those practitioners to diagnose the severity of those conditions more precisely. Accordingly, there is a need for a system that quantifies joint spacing using three-dimensional medical images.

### SUMMARY

In order to more accurately and precisely diagnose conditions affecting joint spacing, a joint space quantification system is disclosed that identifies each bone in a three-dimensional medical image, generates a three-dimensional computer model that includes a three-dimensional representation of each bone, and identifies bone distances (e.g., shortest distances, centroid distances, etc.) between each three-dimensional representation. The joint space quantification system may then identify conditions affecting joint spacing (and quantify the severity of those conditions), for example by comparing the identified bone distances to previous bone distances of the patient and/or the bone distances of patients diagnosed with conditions affecting joint spacing.

In some embodiments, the joint space quantification system also includes a neural network that combines those bone distances with biological, biomechanical, and/or performance data to generate a multivariate model for identifying, predicting, and/or avoiding those conditions affecting joint spacing.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a diagram of an architecture of a joint space quantification system according to an exemplary embodiment.

FIG. 2 is a block diagram of the joint space quantification system according to an exemplary embodiment.

FIG. 3A is a diagram of an example set of bones.

FIG. 3B is an example bone model of carpal bones according to an exemplary embodiment.

FIG. 4A are example bone distances calculated using the example bone model of FIG. 3B according to an exemplary embodiment.

FIG. 4B are additional example bone distances calculated using the example bone model of FIG. 3B according to an exemplary embodiment.

FIG. 5A is a diagram illustrating the quantification of joint spaces by calculating bone distances between each representation in the bone model of FIG. 3B according to an exemplary embodiment.

FIG. 5B is another diagram illustrating the quantification of joint spaces by calculating bone distances between each representation in the bone model of FIG. 3B according to an exemplary embodiment.

FIG. 5C is another diagram illustrating the quantification of joint spaces by calculating bone distances between each representation in the bone model of FIG. 3B according to an exemplary embodiment.

FIG. 5D is another diagram illustrating the quantification of joint spaces by calculating bone distances between each representation in the bone model of FIG. 3B according to an exemplary embodiment.

FIG. 5E is another diagram illustrating the quantification of joint spaces by calculating bone distances between each representation in the bone model of FIG. 3B according to an exemplary embodiment.

FIG. 5F is another diagram illustrating the quantification of joint spaces by calculating bone distances between each representation in the bone model of FIG. 3B according to an exemplary embodiment.

FIG. 5G is another diagram illustrating the quantification of joint spaces by calculating bone distances between each representation in the bone model of FIG. 3B according to an exemplary embodiment.

FIG. 5H is another diagram illustrating the quantification of joint spaces by calculating bone distances between each representation in the bone model of FIG. 3B according to an exemplary embodiment.

FIG. 5I is another diagram illustrating the quantification of joint spaces by calculating bone distances between each representation in the bone model of FIG. 3B according to an exemplary embodiment.

FIG. 5J is another diagram illustrating the quantification of joint spaces by calculating bone distances between each representation in the bone model of FIG. 3B according to an exemplary embodiment.

FIG. 5K is another diagram illustrating the quantification of joint spaces by calculating bone distances between each representation in the bone model of FIG. 3B according to an exemplary embodiment.

FIG. 6A is a block diagram of a neural network trained to generate a multivariate regression model according to an exemplary embodiment.

FIG. 6B is a block diagram of a neural network trained to generate a multivariate classification model according to another exemplary embodiment.

FIG. 6C is a block diagram of a neural network trained to generate another multivariate regression model according to another exemplary embodiment.

FIG. 6D is a block diagram of a neural network trained to generate another multivariate classification model according to another exemplary embodiment.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

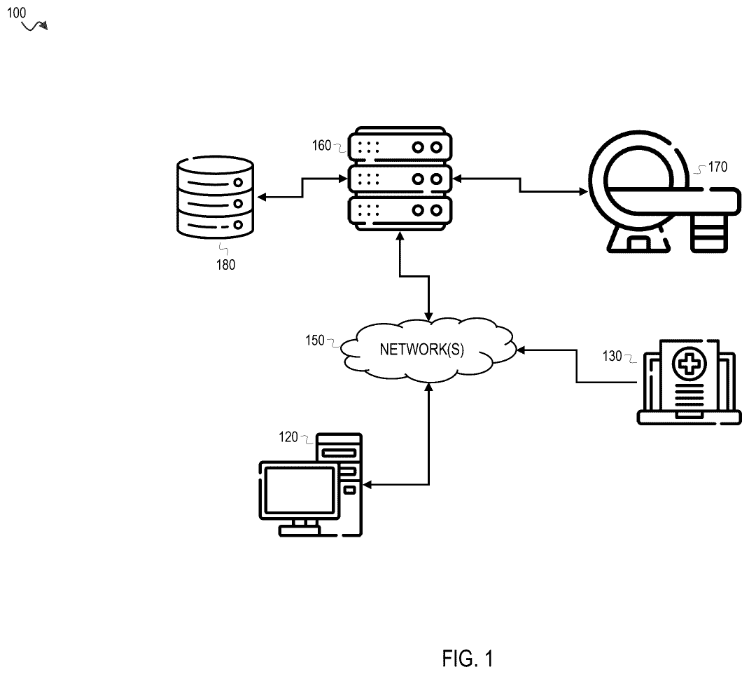

FIG. 1 is a diagram of an architecture 100 of a joint space quantification system 200 according to an exemplary embodiment.

In the embodiment of FIG. 1, the architecture 100 includes a server 160 and non-transitory computer readable storage media 180 in communication with a computing device 120 via one or more computer networks 150. The server 160 and the computing device 120 both include non-transitory computer readable storage media that stores instructions and a hardware computer processing unit that executes those instructions. The server 160 may be any hardware computing device (e.g., a web server, an application server, etc.) suitably configured to perform the functions described herein.

As described in detail below, the server 160 receives medical images (e.g., via the one or more computer networks 150) from a medical imaging system 170, an electronic medical records system 130, etc., and outputs information to a user via a graphical user interface (provided, for example, by the computing device 120). In other embodiments, the medical images may be received by the computing device 120, which performs the functions described herein (e.g., without the use of a server 160).

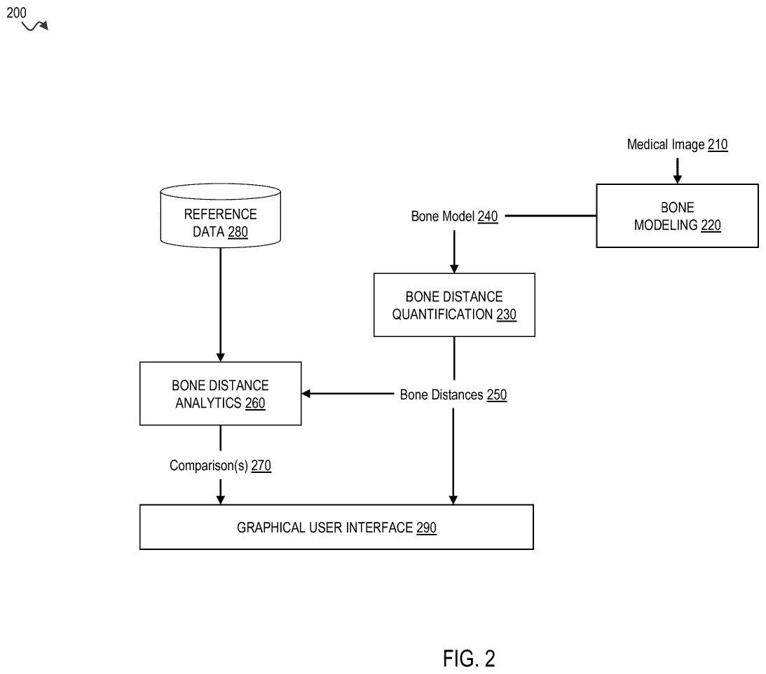

FIG. 2 is a block diagram of the joint space quantification system 200 according to an exemplary embodiment.

As shown in FIG. 2, the joint space quantification system 200 includes a bone modeling unit 220, a bone distance quantification unit 230, and a graphical user interface 290. In some embodiments, the joint space quantification system 200 also includes reference data 280 and a bone distance analytics unit 260. The bone modeling unit 220, the bone distance quantification unit 230, the bone distance analytics unit 260, and the graphical user interface 290 may be realized by software instructions (e.g., stored and executed by the server 160 and/or the computing device 120).

The joint space quantification system 200 receives three-dimensional medical images 210 captured by the medical imaging system 170, stored by the electronic medical records system 130, etc. The medical images 210 may be captured, for example, using radiography, ultrasonography, computed tomography (CT), magnetic resonance imaging (MRI), radiation therapy, etc. The medical images 210 may be stored in any format, for example using the Digital Imaging and Communications in Medicine (DICOM) standard, the Nearly Raw Raster Data (NRRD) format, etc.

Each three-dimensional medical image 210 is an image of a body part that includes a plurality of bones. The bone modeling unit 220 identifies each bone in the medical image 210 and generates a three-dimensional computer model (a bone model 240) of each bone in the medical image 210. The bone model 240 may be stored as an STL file (referred to by various sources as standard triangle language, stereolithography language, and stereolithography tessellation language). The bone modeling unit 220 may generate the bone model 240 by segmenting a DICOM image file (the medical image 210) and exporting the segmented DICOM image file to an STL file, for example using 3D Slicer (http://www.slicer.org) from the Harvard Medical School Surgical Planning Lab; 3DView (http://www.rmrsystems.co.uk/volume_rendering.htm) from RMR Systems Ltd. of East Anglia, UK; Image J (https://imagej.nih.gov/ij) from the U.S. National Institutes of Health; InVesalius (https://invesalius.github.io) from the Renato Archer Information Technology Centre of Sao Paulo, Brazil; Mimics (https://www.materialise.com/en/medical/mimics-innovation-suite/mimics) from Materialise of Leuven, Belgium; the Medical Imaging Interaction Toolkit (http://mitk.org) from the German Cancer Research Center of Heidelberg, Germany; OsiriX (http://www.osirix-viewer.com) from Pixmeo SARL of Geneva, Switzerland; Seg3D (http://www.sci.utah.edu/cibc-software/seg3d.html) from the Scientific Computing and Imaging Institute of Salt Lake City, Utah; Volume Extractor (http://www.i-plants.jp/hp/products/ve3) from Plants Systems of Iwate, Japan; etc.

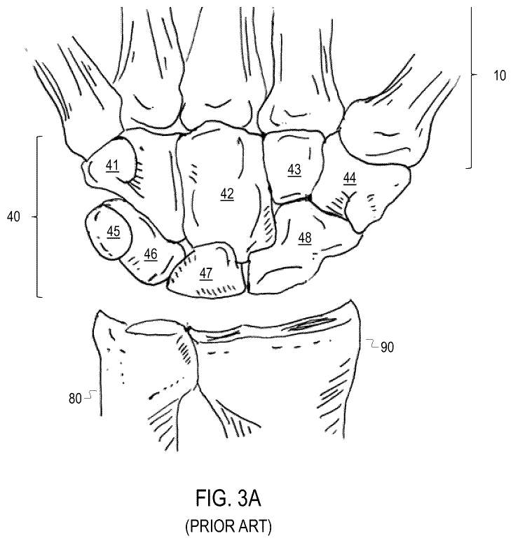

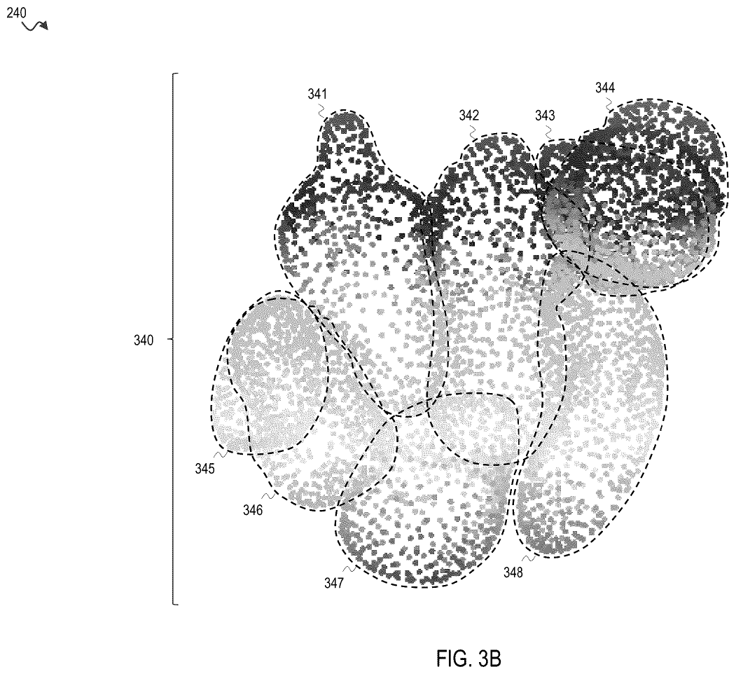

FIG. 3A is a diagram of an example set of bones 40 (in this example, the carpal bones of a human hand) along with the metacarpal bones 10, the ulna 80, and the radius 90. As shown in FIG. 3A, the carpal bones 40 include the hamate bone 41, the capitate bone 42, the trapezoid bone 43, the trapezium bone 44, the pisiform bone 45, the triquetrum bone 46, the lunate bone 47, and the scaphoid bone 48.

FIG. 3B is an example bone model 240 of the carpal bones 40 according to an exemplary embodiment. As shown in FIG. 3B, the bone model 240 includes three-dimensional representations 340 indicative of the size, shape, and relative location of each bone 40 in the medical image 210. In the example of FIG. 3B, for instance, the bone model 240 includes representations 340 of the hamate bone 341, the capitate bone 342, the trapezoid bone 343, the trapezium bone 344, the pisiform bone 345, the triquetrum bone 346, the lunate bone 347, and the scaphoid bone 348.

Referring back to FIG. 2, the bone distance quantification unit 230 measures the distances (the bone distances 250) between each representation 340 of each bone 40 in the bone model 240. The bone distances 250 may include, for example, the shortest distance between each representation 340 of each bone 40 in the bone model 240, the centroid distance between the centroids of each representation 340 of each bone 40 in the bone model 240, etc. Because the bone model 240 is generated using a three-dimensional medical image 210, the bone distances 250 between each representation 340 of each bone 40 in the bone model 240 are indicative of the joint spacing between each bone 40 in the three-dimensional medical image 210.

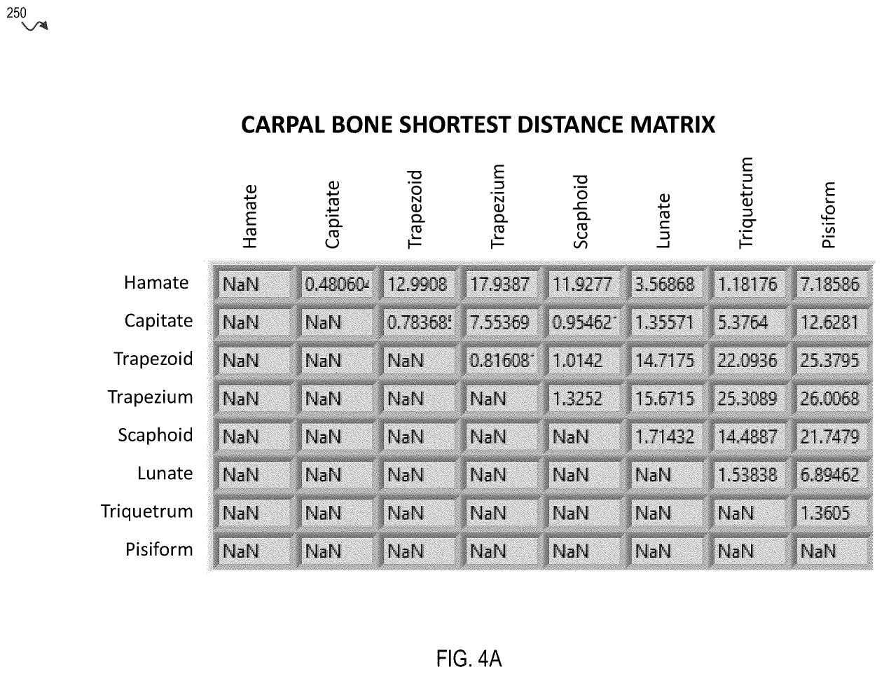

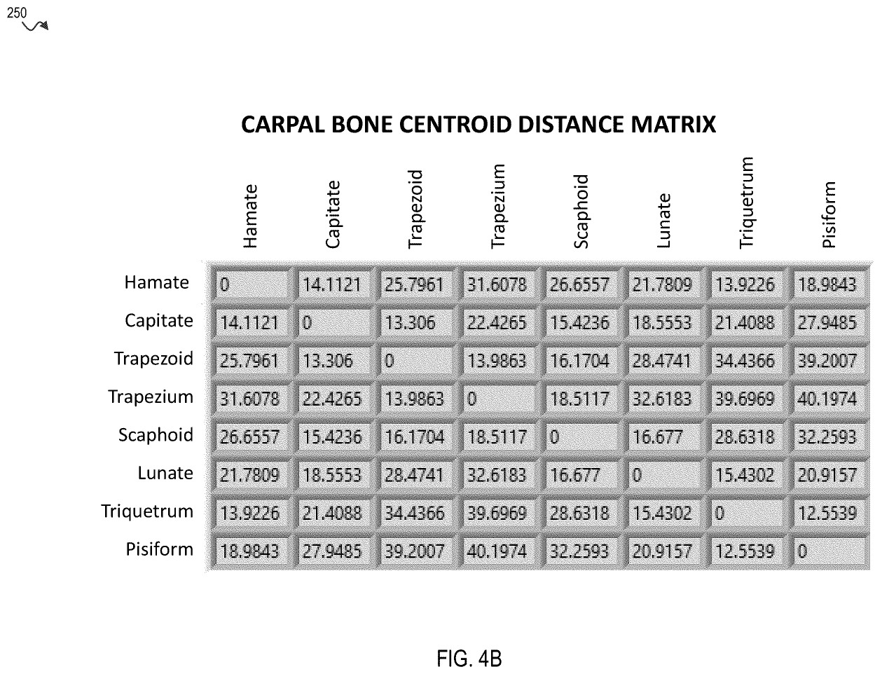

FIGS. 4A and 4B are example bone distances 250 calculated using the example bone model 240 of FIG. 3B according to an exemplary embodiment. In the example of FIG. 4A, the bone distances 250 include the shortest distance between each representation 340 of each bone in the bone model 240. In the example of FIG. 4B, the bone distances 250 include the centroid distances between the centroids of each representation 340 of each bone 40 in the bone model 240.

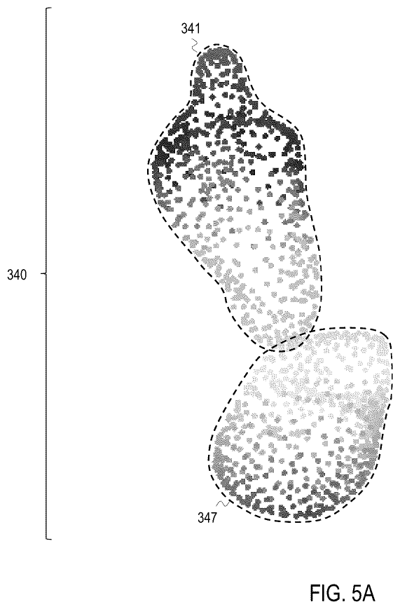

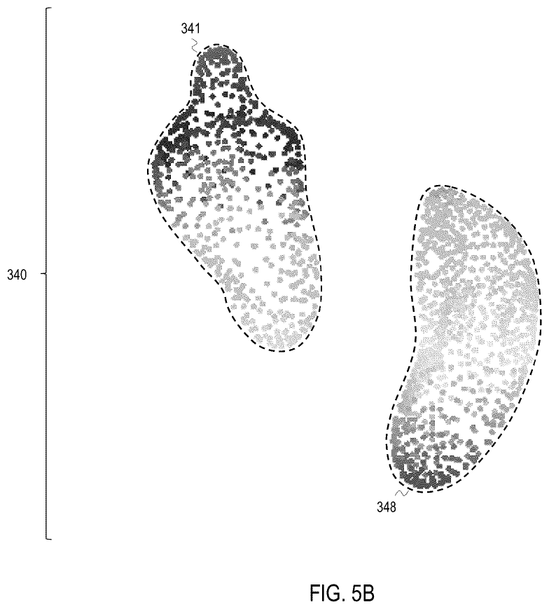

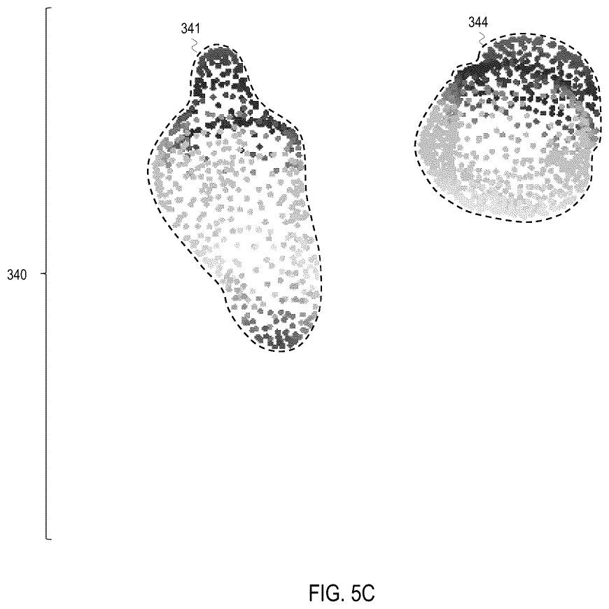

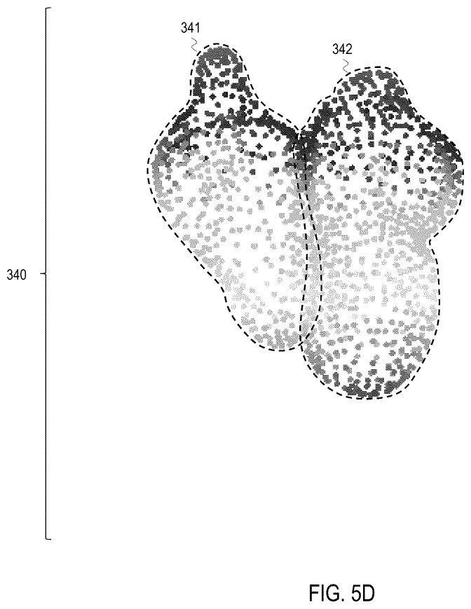

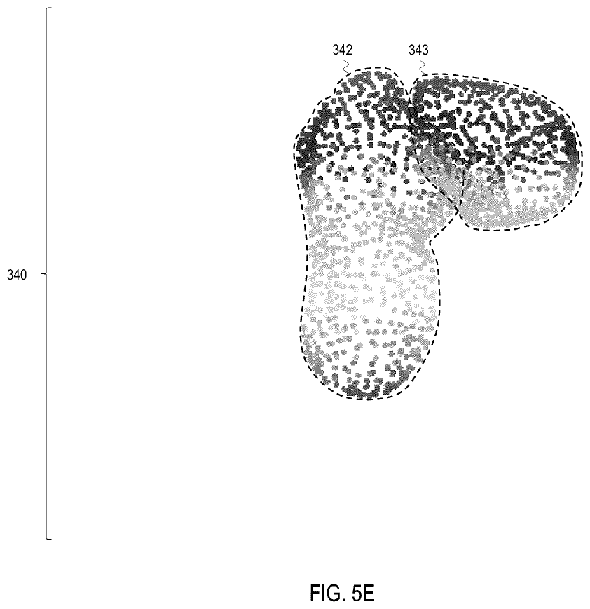

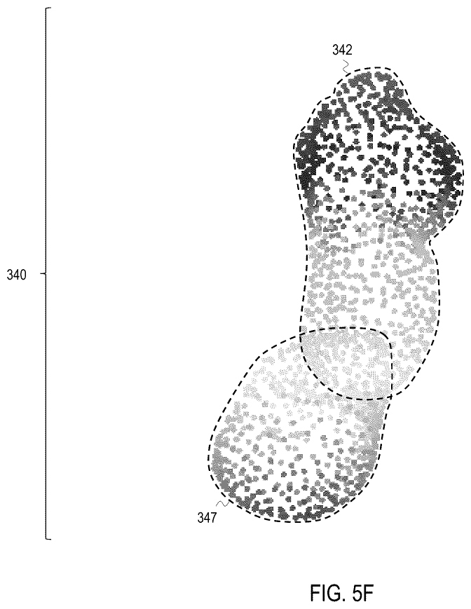

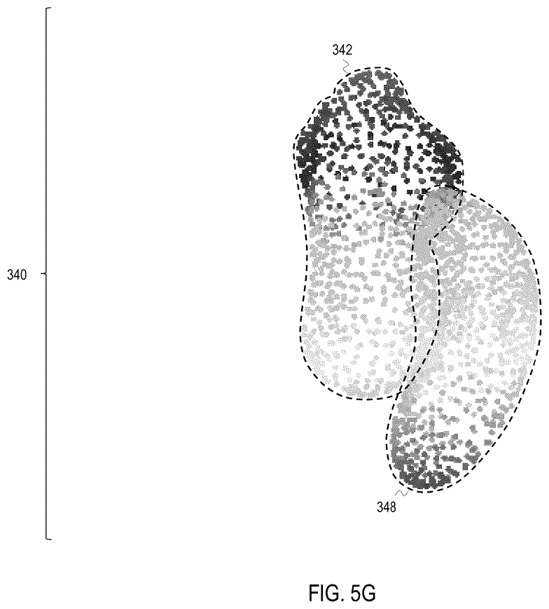

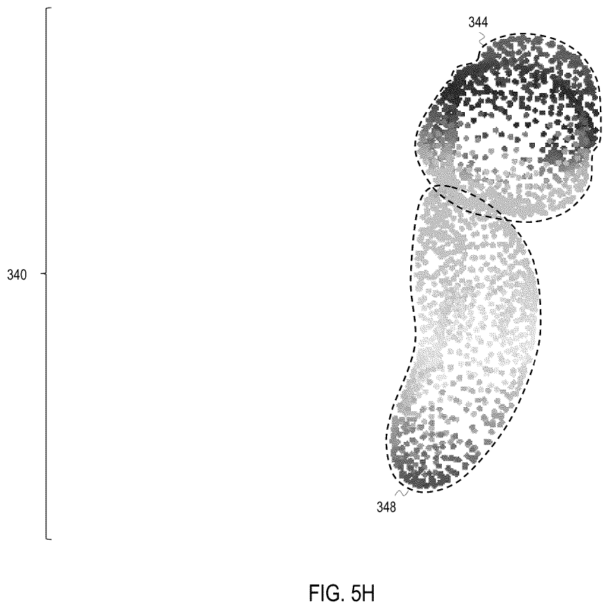

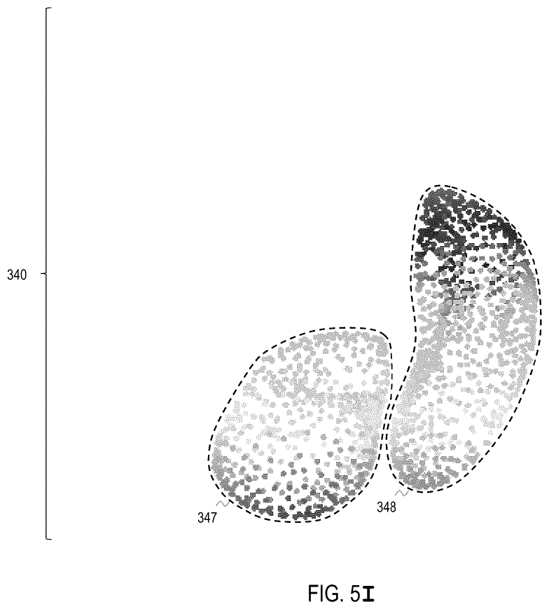

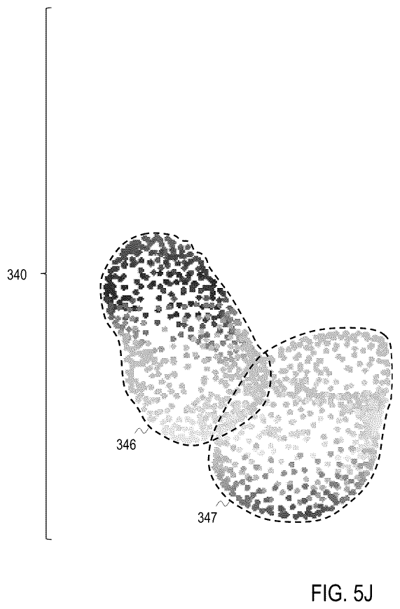

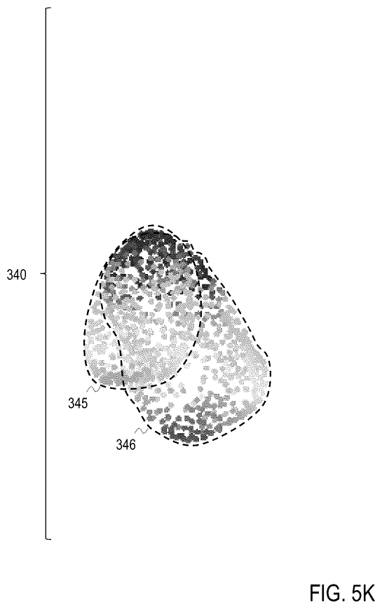

FIGS. 5A through 5K illustrate how the bone distance quantification unit 230 quantifies the joint spaces between each bone 40 in the medical image 210 by calculating the bone distances 250 between each representation 340 of each bone 40 in the bone model 240 according to exemplary embodiments. Using the example bone model 240 of FIG. 3B, for instance, the bone distance quantification unit 230 measures the bone distances 250 (e.g., the shortest distances and/or the centroid distances) between the representations 340 of the hamate bone 341 and the lunate bone 347 (as shown in FIG. 5A), the hamate bone 341 and the scaphoid bone 348 (as shown in FIG. 5B), the hamate bone 341 and the trapezium bone 344 (as shown in FIG. 5C), the hamate bone 341 and the capitate bone 342 (as shown in FIG. 5D), the capitate bone 342 and the trapezoid bone 343 (as shown in FIG. 5E), the capitate bone 342 and the lunate bone 347 (as shown in FIG. 5F), the capitate bone 342 and the scaphoid bone 348 (as shown in FIG. 5G), the scaphoid bone 348 and the trapezium bone 344 (as shown in FIG. 5H), the scaphoid bone 348 and the lunate bone 347 (as shown in FIG. 5I), the lunate bone 347 and the triquetrum bone 346 (as shown in FIG. 5J), the triquetrum bone 346 and the pisiform bone 345 (as shown in FIG. 5J), etc.

The shortest distance between any two bones 40 can be determined by capturing a two-dimensional medical image along an axis that is orthogonal to the shortest vector between those two bones 40 and measuring the length of that vector. However, to calculate the shortest distance between more than two bones 40 using only two-dimensional images, a two-dimensional image must be captured orthogonal to each of the shortest vectors between each two bones 40. This becomes even more difficult when attempting to calculate the shortest distances between a set of bones 40 that overlap when viewed along any axis that is orthogonal to the shortest vector between any two of those bones 40 (like the carpal bones 40 shown in FIGS. 4B and 5A through 5K). The joint space quantification system 200 overcomes that issue by using a three-dimensional medical image 210 and generating a three-dimensional bone model 240 that includes a three-dimensional representation 340 of each bone 40 in the medical image 210, enabling the bone distance quantification unit 230 to measure the shortest bone distance 250 between each bone 40 in the medical image 210 by measuring the shortest bone distance 250 between each representation 340 of each bone 40 in the bone model 240. Using a three-dimensional medical image 210 and generating three-dimensional representations 340 of each bone 40 also enables the bone distance quantification unit 230 to identify the centroid of each three-dimensional representation 340, which would not be possible using two-dimensional medical images, and measure the centroid bone distance 250 between each bone 40 in the medical image 210 by measuring the centroid bone distance 250 between each centroid of each representation 340 of each bone 40 in the bone model 240.

Referring back to FIG. 2, the bone distances 250 are output via the graphical user interface 290. Accordingly, instead of simply looking at medical images 210 and subjectively diagnosing conditions affecting joint spacing (and qualitatively diagnosing the severity of those conditions), the joint space quantification system 200 enables practitioners to view the precise bone distances 250 between the bones 40 in the medical image 210, enabling those practitioners to diagnose conditions affecting joint spacing earlier and more accurately (while also diagnosing the severity of those conditions more precisely).

In some embodiments, the joint space quantification system 200 also includes a bone distance analytics unit 260 that compares the bone distances 250 (calculated by measuring the distances between each representation 340 of each bone 40 in a medical image 210) to reference data 280 to generate one or more comparisons 270, which are output via the graphical user interface 290. In some embodiments, for instance, the reference data 280 may include bone distances 250 calculated using previously captured medical images 210 of the same patient, enabling the bone distance analytics unit 260 to calculate changes in bone distances 250 over time. In those embodiments, practitioners could predict the onset of conditions affecting joint spacing or monitor the severity of conditions affecting joint spacing after diagnosis.

In some embodiments, the reference data 280 may include datasets of the bone distances 250 of patients having been diagnosed with conditions affecting joint spacing. For example, ligaments of cadavers may be cut (to form a partial tear, a complete tear, etc.) and the joint space quantification system 200 may be used to measure the bone distances 250 of the cadavers and/or the changes in bone distances 250 before and after the ligament injury. Additionally or alternatively, patients with conditions affecting joint spacing may be diagnosed (and the severity of those conditions may be characterized) and the joint space quantification system 200 may be used to measure the bone distances 250 in the medical images 210 of patients with those diagnosed conditions. For instance, surgeons having viewed and attempted to repair damaged ligaments during surgery may identify and characterize the severity of ligament injuries. In other instances, autopsies of diseased patients may be performed to identify and characterize the severity of conditions affecting joint spacing.

Using the bone distances 250 of patients having been diagnosed with conditions affecting joint spacing, the bone distance analytics unit 260 may identify thresholds for diagnosing those conditions (and/or thresholds for characterizing the severity of those conditions) and compare the bone distances 250 identified in the medical images 210 to those thresholds. Additionally or alternatively, the bone distance analytics unit 260 may use machine learning or artificial intelligence to quantitatively assess the bone distances 250 of an individual as described below.

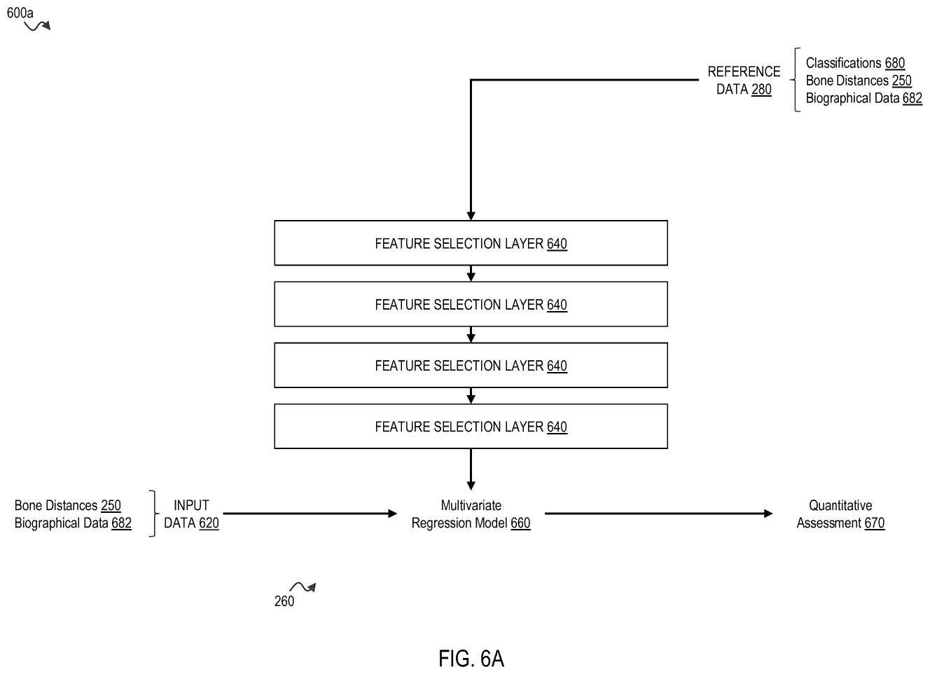

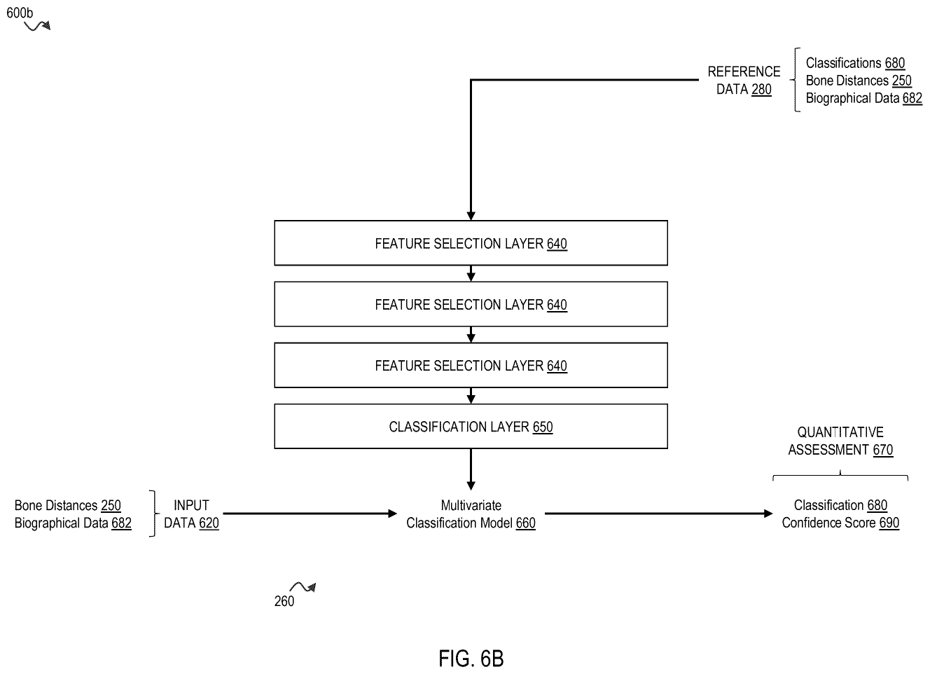

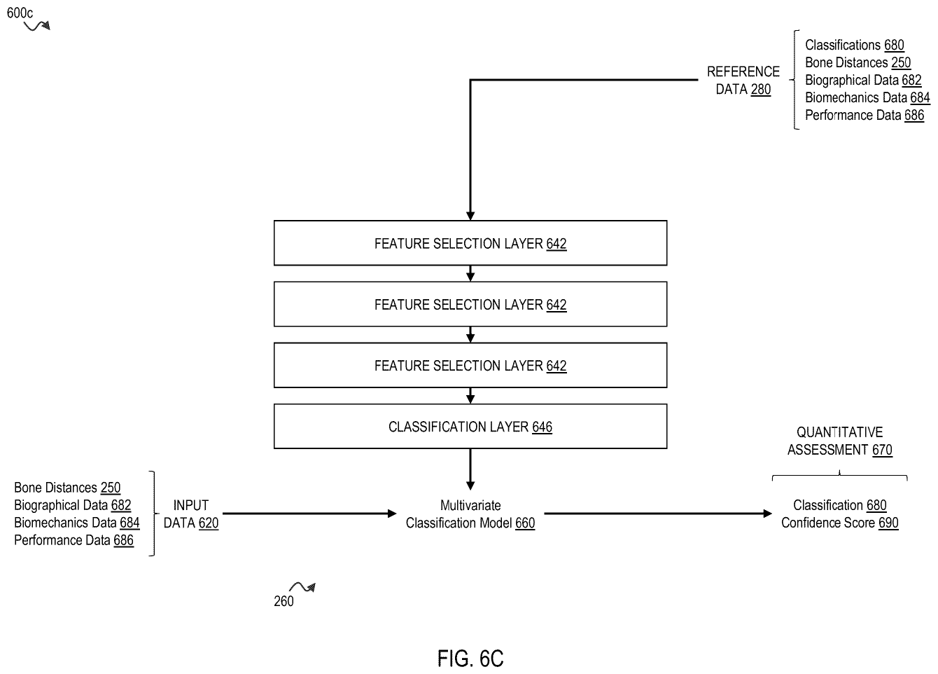

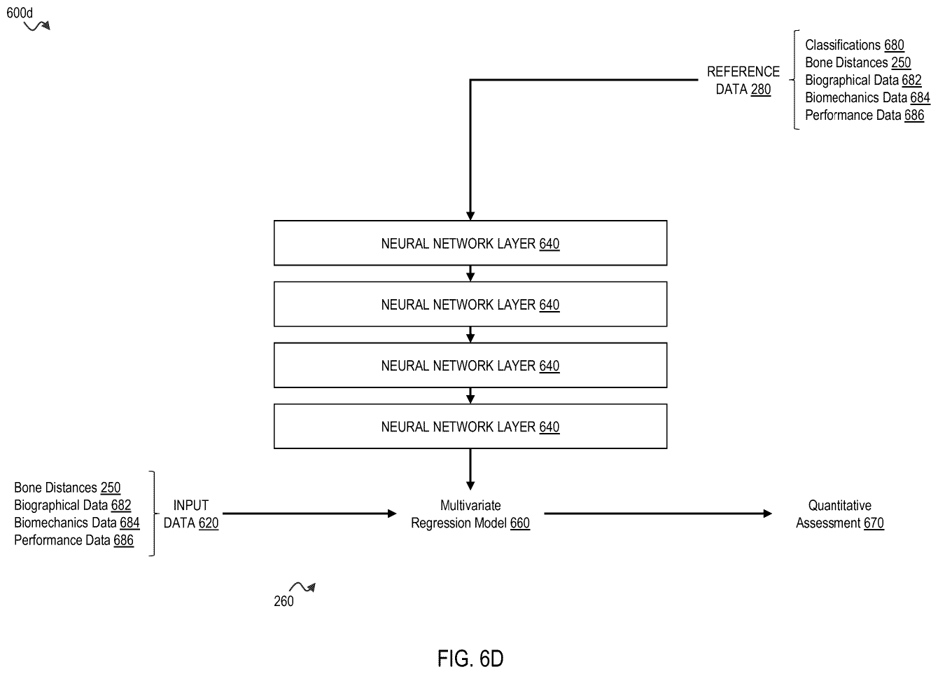

FIGS. 6A through 6D are block diagrams of a neural network (individually referred to in various embodiments as neural networks 600a through 600d) trained using the reference data 280 to generate a multivariate model 660 for diagnosing conditions affecting joint spacing according to exemplary embodiments.

As shown in FIG. 6A, a neural network 600a may be trained using the reference data 280 to generate a multivariate regression model 660 used to generate a quantitative assessment 670. The quantitative assessment 670 may be, for example, a numerical score calculated using the bone distances 250 of a patient (referred to as input data 620) indicative of the likelihood that the patient has a condition affecting joint spacing (or more than one numerical score indicative of the likelihoods that the patient has various conditions affecting joint spacing). As shown in FIG. 6A, in some embodiments, the reference data 280 and the input data 620 may also include biological data 682 (e.g., age, height, weight, demographic information, etc., received, for example, from the electronic medical records system 130) that, when combined with the bone distances 250, make the multivariate regression model 660 (and by extension, the quantitative assessments 670) more accurate in predicting various conditions affecting joint spacing.

To generate the multivariate regression model 660, the neural network 600a may include a number of feature selection layers 640 that identify features in the bone distances 250 included in the reference data 280 indicative of potential injuries affecting joint spacing and weights those features in accordance their correlation with potential injuries relative to the other features in the reference data 280. For example, the neural network 600a may be trained using supervised learning where each set of bone distances 250 in the reference data 280 includes a classification 680 indicative of whether those bone distances 250 were captured from a patient diagnosed with an injury affecting bone spacing (and, in some embodiments, an assessment of the severity of that injury).

The feature selection layers 640 reduce the number of input variables to those that are believed to be most useful to predict the classification 680. Accordingly, the feature selection layers 640 remove non-informative or redundant predictors from the multivariate regression model 660, reducing the amount of system memory required generate and execute the multivariate regression model 660 and improving performance by removing input variables that are not relevant to the classification 680 (and can add uncertainty to the predictions and reduce the overall effectiveness of the multivariate regression model 660). For example, the feature selection layers 640 may perform a filter feature selection method, which use statistical techniques to evaluate the relationship between each input variable and the target variable and use those scores to choose and weight the input variables are used in the multivariate regression model 660. Alternatively, the feature selection layers 640 may perform wrapper feature selection (e.g., recursive feature elimination) by creating many multivariate models with different subsets of input features, evaluating each of those models by adding and removing potential predictors, and selecting the best performing model according to a performance metric. In yet another example, the feature selection layers 640 may use an intrinsic feature selection method (e.g., penalized regression models such as Lasso, decision trees, or ensembles of decision trees such as random forest). To identify and weight each of the features, the feature selection layers 640 may, for example, identify correlation coefficients (e.g., Pearson's correlation coefficients) for linear correlations or a rank-based methods (e.g., Spearman's rank coefficients) for nonlinear correlations.

As shown in FIG. 6B, a neural network 600b may be trained using the reference data 280 to generate a multivariate classification model 660 used classify the input data 620 of a patient (including the bone distances 250 of the patient and, in some embodiments, biographical data 682 as described above) as being most likely to have a certain classification 680 (and, in some embodiments, confidence score 690 indicative of the probability of that the classification 690 identified by the multivariate classification model 660 is accurate). In the embodiments of FIG. 6B, in addition to one or more feature selection layers 640 as described above, the neural network 600b may include one or more classification layers 650 trained using the reference data 280 to classify the input data 620 based on features identified in the input data 620 and the correlations, identified by the feature selection layers 640, with the classifications 680 in the reference data 280. To train the neural network 600b to identify those correlations, for example, the neural network 600b may be configured to calculate (analysis of variance (ANOVA) correlation coefficients for linear correlations or Kendall's rank coefficients for nonlinear correlations.

As shown in FIGS. 6C and 6D, the neural network 600c or 600d may be trained using reference data 280 that further includes biomechanics data 684 and/or performance data 686 to generate a multivariate regression or classification model 660 that calculates a quantitative assessment using input data 220 that, similarly, also includes biomechanics data 684 and/or performance data 686 of the patient. As described below, the biomechanics data 684 may include any information indicative of the movement of one or more body parts and, in some instances, the structure of those body parts). The biomechanics data 684 may be captured, for example, using motion capture technology (e.g., Hawk-Eye, KinaTrax, Theia3D, Simi, etc.) The performance data 686 may include any quantitative information indicative of an amount of physical exertion (e.g., the number of exercises performed during physical therapy and the weight, speed, time, distance, etc., of those exercises) and an amount of rest between those physical exertions. Additionally or alternatively, the performance data 686 may include quantitative information indicative of the results of those physical exertions, such as information captured during a sporting event (for example, using Hawk-Eye, Pitchf/x, TrackMan, etc.) indicative of the quality of the individual's performance.

In the embodiments of FIGS. 6A-6D, the neural network 600 may be trained and executed by the bone distance analytics unit 260. For instance, the server 160 may train the neural network 600 to generate the multivariate model 660 using the reference data 280 and either the server 160 or the computing device 120 may use the multivariate model 660 to generate a quantitative assessment 670 based on the input data 620.

The joint space quantification system 200 may be used to help individuals recover from or avoid conditions affecting joint spacing. For example, physical therapy providers may capture medical images 210 from patients, use the joint space quantification system 200 to quantify and monitor the bone distances 250 of each patient, and develop or adjust exercise routines that minimize recovery time and the likelihood of reinjury. In another example, a professional baseball organization may periodically capture medical images 210 of pitcher's arms, use the joint space quantification system 200 to quantify the monitor the bone distances 250 of each pitcher, and develop or adjust pitching schedules (and other training schedules) to minimize ligament damage and other arm injuries while enabling pitchers to build up arm strength and/or recover from arm injuries.

The joint space quantification system 200 may also be used to identify individuals with potential injuries even before medical images 210 have even been captured. For instance, a multivariate model 660 may be used to identify biomechanics data 684 and/or performance data 686 that is indicative of an individual having suffered an injury affecting joint spacing. Returning to the baseball example above, an organization may use the joint space quantification system 200 to identify players having biomechanics data 684 and/or performance data 686 indicative of an injury and respond by capturing medical images 210 of those players so they can be analyzed (qualitatively by a medical practitioner and/or quantitatively using the joint space quantification system 200) before the player inadvertently causes further damage.

Finally, the joint space quantification system 200 may also be used to identify and avoid biomechanical activities that may cause injuries affecting joint spacing. For instance, a multivariate model 660 may be used to identify biomechanics data 684 that is correlated with individuals later suffering an injury affecting joint spacing. In the baseball example above, for instance, an organization may use the joint space quantification system 200 to both identify biomechanics data 684 (in the reference data 280) that is correlated with future injury and identify players, using the multivariate model 660, having biomechanics data 684 indicative of those injuries. Using that information, the organization may then intervene before the player inadvertently causes that injury.

While preferred embodiments of the joint space quantification system 200 have been described above, those skilled in the art who have reviewed the present disclosure will readily appreciate that other embodiments can be realized within the scope of the invention. Accordingly, the present invention should be construed as limited only by any appended claims.

What is claimed is:

1. A method, comprising:
receiving a three-dimensional medical image of a body part that includes a plurality of bones;
identifying each of the bones in the three-dimensional medical image;
generating a three-dimensional computer model that includes a three-dimensional representation of each bone identified in the three-dimensional medical image; and
identifying bone distances between each bone in the body part by measuring the distances between each three-dimensional representation of each bone.

2. The method of claim 1, wherein the three-dimensional medical image is a computed tomography (CT) scan or a magnetic resonance image (MM).

3. The method of claim 1, wherein the bone distances are the shortest distances between each three-dimensional representation of each bone or the centroid distances between the centroids of each three-dimensional representation of each bone.

4. The method of claim 1, further comprising:
comparing the bone distances to reference data.

5. The method of claim 4, wherein the reference data includes the bone distances identified using a previous medical image of the body part.

6. The method of claim 4, wherein the reference data includes thresholds generated by analyzing the bone distances of patients diagnosed with conditions affecting joint spacing.

7. The method of claim 4, wherein comparing the bone distances to the reference data comprises applying a multivariate model generated by a neural network trained using the bone distances and biological data of patients diagnosed with conditions affecting joint spacing.

8. The method of claim 7, wherein the biological data includes age, height, or weight.

9. The method of claim 7, wherein the machine learning model is also trained using biomechanics data of the patients diagnosed with conditions affecting joint spacing.

10. The method of claim 1, wherein at least two of the plurality of bones overlap when viewed along an axis that is orthogonal to the shortest vector between any two of the plurality of bones.

11. A joint space quantification system, comprising:
non-transitory computer readable storage media that stores a three-dimensional medical image of a body part that includes a plurality of bones; and
a hardware computer processor that:
identifies each of the bones in the three-dimensional medical image;
generates a three-dimensional computer model that includes a three-dimensional representation of each bone identified in the three-dimensional medical image; and
identifies bone distances between each bone in the body part by measuring the distances between each three-dimensional representation of each bone.

12. The system of claim 11, wherein the three-dimensional medical image is a computed tomography (CT) scan or a magnetic resonance image (MM).

13. The system of claim 11, wherein the bone distances are the shortest distances between each three-dimensional representation of each bone or the centroid distances between the centroids of each three-dimensional representation of each bone.

14. The system of claim 11, wherein:
the non-transitory computer readable storage media stores reference data that includes the bone distances identified using a previous medical image of the body part; and
the hardware computer processor compares the bone distances to reference data.

15. The system of claim 14, wherein the reference data includes thresholds generated by analyzing the bone distances of patients diagnosed with conditions affecting joint spacing.

16. The system of claim 14, further comprising:
a neural network trained using the bone distances and biological data of patients diagnosed with conditions affecting joint spacing to generate a multivariate model for calculating a qualitative assessment based on the bone distances identified in the three-dimensional representations.

17. The system of claim 16, wherein the biological data includes age, height, or weight.

18. The system of claim 16, wherein the machine learning model is also trained using biomechanics data of the patients diagnosed with conditions affecting joint spacing.

19. The system of claim 11, wherein at least two of the plurality of bones overlap when viewed along an axis that is orthogonal to the shortest vector between any two of the plurality of bones.

20. Non-transitory computer readable storage media storing instructions that, when executed by a hardware computer processor, cause a computing device to:
identify each of a plurality of bones in a three-dimensional medical image of a body part;
generate a three-dimensional computer model that includes a three-dimensional representation of each bone identified in the three-dimensional medical image; and
identify bone distances between each bone in the body part by measuring the distances between each three-dimensional representation of each bone.
